(19) United States
(12) Patent Application Publication
(10) Pub. No.: US 2024/0384235 A1
(43) Pub. Date: Nov. 21, 2024

(54) BIOMATERIALS CONTAINING UMBILICAL CORD-DERIVED STEM CELLS

(71) Applicant: GLOBUS MEDICAL, INC., AUDUBON, PA (US)

(72) Inventors: Bipin Gaihre, Malvern, PA (US); Archana Bhat, Phoenixville, PA (US); Jason Leigl, Collegeville, PA (US); Joshua Cige, Conshohocken, PA (US)

(21) Appl. No.: 18/320,499

(22) Filed: May 19, 2023

(51) Int. Cl. C12N 5/0775; A61P 43/00

(52) U.S. Cl. CPC C12N 5/0665; A61P 43/00; C12N 2500/02; C12N 2539/00

(57) ABSTRACT

Biomaterials containing viable stem cells, methods of making the biomaterials, and methods of promoting musculoskeletal tissue healing in a mammal. The stem cell-based regenerative biomaterial may include an active ingredient including umbilical cord-derived mesenchymal stem cells, isolated and expanded at low-oxygen similar to that experienced in degenerative human tissues. This preconditioning enables the injected stem cells to better adapt to the degenerative microenvironment resulting in improved viability.

### FIELD OF THE INVENTION

The present application relates generally to musculoskeletal tissue healing biomaterials containing viable stem cells, methods of making the biomaterials from umbilical cord-derived stem cells, and methods of treatment including promoting tissue healing by administering the biomaterial to a mammal.

### BACKGROUND OF THE INVENTION

Degenerative disc disease (DDD) is a major cause of low back pain and disability worldwide. Degenerative joint osteoarthritis (OA) is a major cause of joint-related pain that may be chronic if not intervened during early disease progression. About one in four adults in the United States report experiencing severe joint pain related to osteoarthritis and more than 50% of these adults experience persistent pain. Both conventional and surgical treatment options are considered palliative approaches to pain management. The major pathological hallmarks of degenerative disc disease and osteoarthritis are the increased catabolic activity and the gradual reduction of functional cells.

Stem cell-based regenerative therapies have been studied with promising results at both pre-clinical and clinical levels. The relatively low-risk and low-cost associated with these therapeutic approaches make them attractive from a clinical and commercial perspective. Despite the positive results, at pre-clinical stage and at early stage-clinical stage, of stem cells for the treatment of early-stage degenerative disc disease and osteoarthritis repair, low cell survival due to the hypoxic and inflammatory host environment, cell leakage from the injection site resulting in osteophyte formation, and/or the identification of optimal cell source and development of optimal cell carrier still present major challenges to stem cell therapy for these degenerative diseases. Addressing these needs are imperative to the development of viable stem cell therapies with durable therapeutic outcomes.

### SUMMARY OF THE INVENTION

To meet this and other needs, biomaterials described herein may be advantageous for musculoskeletal tissue healing and repair. The biomaterials may be used, for example, for the treatment of early-stage degenerative diseases of intervertebral discs and joints. The stem cell-based regenerative therapy product may include an active ingredient including umbilical cord-derived mesenchymal stem cells, isolated and expanded at low-oxygen similar to that experienced in degenerative human tissues. This preconditioning enables the injected stem cells to better adapt to the degenerative microenvironment resulting in improved viability. In addition, the carrier material may include a viscous gel or a thermoresponsive gel that undergoes physical gelation at physiological temperature, thereby preventing early leakage and diffusion of stem cells from the injection site and focusing the treatment effects on the diseased site.

According to one embodiment, a biomaterial for aiding musculoskeletal tissue regeneration includes human umbilical cord mesenchymal stem cells preconditioned under a hypoxic environment and a thermo-responsive carrier being a viscous liquid at cold temperatures between 2-8° C. (e.g., 4° C.) and a gel at physiological temperature of 37° C.

The biomaterial may include one or more of the following features. The thermo-responsive carrier may include hyaluronic acid, gellan gum, methyl cellulose, or a combination thereof. The thermo-responsive carrier may include a co-dissolution of hyaluronic acid and methyl cellulose. The thermo-responsive carrier may include 0.2% to 2% (w/v) hyaluronic acid and 2-10% (w/v) methyl cellulose. The human umbilical cord mesenchymal stem cells may include a single cell suspension and/or a suspension of micro-spheroids having 50-200 cells with sizes ranging from 50 to 200 μm. The hypoxic conditions may include an oxygen concentration of 10% or less (e.g., about 1-6% oxygen). The human umbilical cord mesenchymal stem cells may be obtained from Wharton's jelly of a human umbilical cord.

According to one embodiment, a method for manufacturing a biomaterial for aiding musculoskeletal tissue regeneration may include: (1) obtaining human umbilical cord; (2) isolating viable stem cells from the umbilical cord; (3) expanding the viable stem cells under hypoxic conditions; and (4) combining the expanded viable stem cells with an inert carrier to form a therapeutic product.

The method may include one or more of the following features. The hypoxic conditions may include an oxygen concentration of 10% or less (e.g., about 1-6% oxygen). The viable stem cells may be mesenchymal stem cells. The viable stem cells may include a single cell suspension, suspension of micro-spheroids, and/or a combination of both. The viable stem cells may be obtained from Wharton's jelly of a human umbilical cord. The inert carrier may include a thermo-gelation agent, such as hyaluronic acid, gellan gum, methyl cellulose, or a combination thereof.

According to one embodiment, a method of promoting musculoskeletal tissue healing in a mammal may include providing a biomaterial including human umbilical cord mesenchymal stem cells preconditioned under a hypoxic environment; and administering the biomaterial into a target repair site to facilitate repair or regeneration of musculoskeletal tissue at the target repair site. The biomaterial may be injected into the target repair site as a liquid that transitions to a gel at physiological temperature. The target repair site may be a defect or injury in the spine or a joint, such as a knee, hip, shoulder, etc.

According to yet another embodiment, a kit includes one or more biomaterials, implants, or components thereof described herein. For example, the kit may contain a vial containing an injectable composition of the biomaterial having viable stem cells preconditioned in a hypoxic environment and a syringe with accessories for injection. The kit may contain biomaterial compositions of the same or different types. In addition, the kit may include other components known in the art, including, but not limited to, carriers or scaffolds, cages or interbody devices (e.g., titanium and/or polyether ether ketone (PEEK) spacers), allograft spacers, cell culture media, phosphate buffered saline (PBS), a tissue culture substrate, bone graft harvesting tools, bone marrow aspirate retrieval tools, or the like.

### BRIEF DESCRIPTION OF THE DRAWINGS

A more complete understanding of the present invention, and the attendant advantages and features thereof, will be more readily understood by reference to the following detailed description when considered in conjunction with the accompanying drawings, wherein:

FIG. 1 shows a process flowchart of the steps for producing a therapeutic product with pre-conditioned viable stem cells according to one embodiment;

FIG. 2 is a cross-sectional view of a human umbilical cord;

FIG. 3 shows images of isolation and expansion of stem cells under hypoxic conditions at passage 0 (P0), passage 1 (P1), and passage 2 (P2), respectively;

FIG. 4 shows images of expansion of stem cells under normoxic conditions at passage 1 (P1) and passage 2 (P2), respectively;

FIG. 5 is a graph showing the flow cytometry characterization for the surface markers of the stem cells isolated and expanded in normoxic, hypoxic, and isolated in normoxic and expanded in hypoxic conditions, respectively;

FIGS. 6A-6B show graphs with the comparative analysis of proliferation of stem cells under hypoxic and normoxic condition at different passage levels (P0, P1, P2, P3), respectively;

FIG. 7 is a graph of a real time polymerase chain reaction (PCR) study demonstrating the upregulation of nucleus specific markers (SOX9 and COL2a) after hypoxic preconditioning compared to normoxic cultured stem cells;

FIG. 8 shows carrier development and optimization with hyaluronic acid (HA) and methyl cellulose (MC) in different amounts;

FIGS. 9A-9G show injection of carrier materials to ex vivo spine and evaluation under static and mechanical loading;

FIG. 10 shows a process flowchart of the steps for producing a therapeutic product including micro-spheroids according to further embodiments; and

FIG. 11 shows a process flowchart of the steps for producing a therapeutic product including cultured cells according to further embodiments.

### DETAILED DESCRIPTION OF THE INVENTION

The present application relates generally to musculoskeletal tissue healing biomaterials containing pre-conditioned viable stem cells, methods of making the biomaterials from umbilical cord-derived stem cells, methods of treatment including promoting musculoskeletal tissue healing by administering the biomaterial to a mammal, and kits thereof. The stem cells may be isolated and/or expanded in vitro under hypoxic conditions for the stem cells to precondition to a hypoxic microenvironment. This enables the pre-conditioned cells to adapt and maintain their functions within the host injection site, thereby resulting in improved viability and therapeutic efficacy.

One of the active ingredients may include umbilical cord-derived mesenchymal stem cells, isolated and/or expanded at low-oxygen conditions similar to that experienced in degenerative human tissues. The preconditioning enables the injected stem cells to better adapt to the degenerative microenvironment resulting in improved viability. This may help to stop the degenerative cascade through the paracrine effects and/or regeneration of new tissues through the engraftment to host tissue. Furthermore, the carrier material may include a viscous gel or thermoresponsive system that undergoes physical gelation at physiological temperature, thereby effectively administering the treatment effects to the diseased site and preventing early leakage and diffusion of stem cells from the injection site.

Additional aspects, advantages and/or other features of example embodiments of the invention will become apparent in view of the following detailed description. It should be apparent to those skilled in the art that the described embodiments provided herein are merely exemplary and illustrative and not limiting. Numerous embodiments of modifications thereof are contemplated as falling within the scope of this disclosure and equivalents thereto.

In describing example embodiments, specific terminology is employed for the sake of clarity. However, the embodiments are not intended to be limited to this specific terminology. Unless otherwise noted, technical terms are used according to conventional usage. As used herein and in the claims, the terms “comprising” and “including” are inclusive or open-ended and do not exclude additional unrecited elements, compositional components, or method steps. Accordingly, the terms “comprising” and “including” encompass the more restrictive terms “consisting essentially of” and “consisting of.”

Unless specified otherwise, all values provided herein include up to and including the endpoints given, and the values of the constituents or components of the compositions are expressed in weight percent or % by weight of each ingredient in the composition. Each compound used herein may be discussed interchangeably with respect to its chemical formula, chemical name, abbreviation, etc. For example, HA may be used interchangeably with hyaluronic acid. Embodiments described herein may be generally directed to biomaterials, implants made therefrom, methods of making the same, and methods of using the same to promote musculoskeletal tissue healing.

The composition may be “biocompatible” as that term refers to the ability (e.g., of a composition or material) to perform with an appropriate host response in a specific application, or at least to perform without having a toxic or otherwise deleterious effect on a biological system of the host, locally or systemically. The biomaterial may be “biologically degradable” in that the material may be degraded by cellular absorption and/or hydrolytic degradation in a patient's body after a given duration, for example.

According to one embodiment, the biomaterial composition may be configured to facilitate repair or regeneration of musculoskeletal tissue at a target repair site or treatment site. The target repair site or treatment site may be, for example, an area between musculoskeletal tissues including, soft tissue, a joint, another bony structure, or a void, gap, defect, or surgeon created opening in the musculoskeletal tissue. For example, the biomaterial composition may be configured to facilitate musculoskeletal tissue healing at a target repair site in the spine, facet and sacroiliac joint, pelvis, the cranium, an extremity, knee, hip, shoulder, rotator cuff, etc., or another musculoskeletal tissue, between musculoskeletal tissues, or bony structure in the patient's body. The biomaterial composition may be configured to be directly injected or otherwise disposed at or in contact with the target repair site or treatment site. The patient and target repair site may be in a human, mammal, or other organism.

The biomaterials may be used, for example, for the treatment of early-stage degenerative diseases at intervertebral discs and joints. The stem cell-based regenerative therapy product may include an active ingredient including umbilical cord-derived mesenchymal stem cells, isolated and/or expanded under hypoxic conditions at low-oxygen concentrations. This preconditioning enables the injected stem cells to better adapt to the degenerative microenvironment resulting in improved viability. In addition, the carrier material may include a viscous gel or a thermoresponsive gel that undergoes physical gelation at physiological temperature, thereby focusing the treatment effects on the diseased site and preventing early leakage and diffusion of stem cells from the injection site.

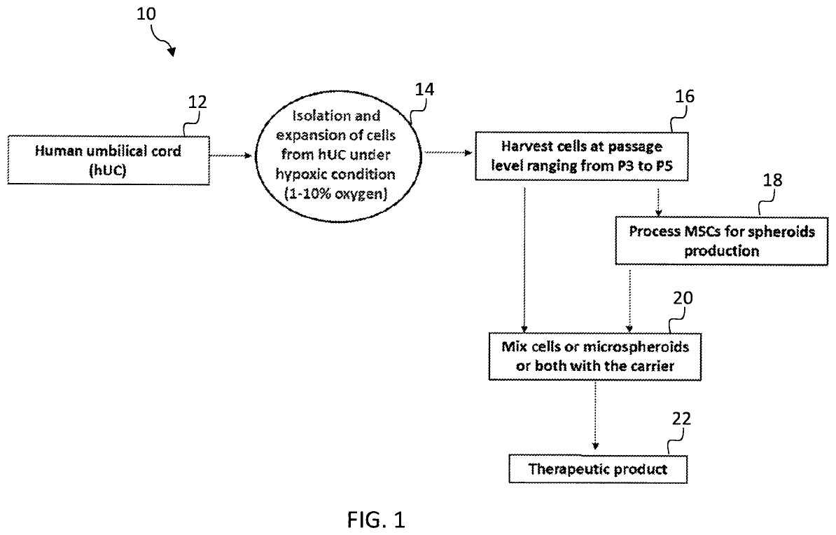

Referring now to FIG. 1, a flowchart of process 10 for preparing a therapeutic product containing viable pre-conditioned stem cells is shown according to one embodiment. The process 10 may include one or more of the following steps: (1) obtaining human umbilical cord (hUC) 12; (2) isolating and expanding stem cells 14 from the human umbilical cord under hypoxic conditions; (3) harvesting pre-conditioned stem cells 16; (4) optionally, processing the pre-conditioned stem cells into spheroids 18; (5) mixing the pre-conditioned cells 16 and/or spheroids 18 with a carrier 20; and (6) obtaining a therapeutic product 22 containing viable stem cells preconditioned under hypoxic conditions.

In step 12, umbilical cord is obtained, for example, from humans or another mammal. Preferably, the umbilical cord is derived from a human subject, although it is envisioned that the umbilical cord may be derived from other sources. The umbilical cord, with or without attached placentas, may be obtained from human volunteer donors. In particular, donors may include mothers who have undergone an elective Caesarian procedure for childbirth. Prior to processing, the umbilical cord is preferably screened for various diseases, illicit drug use, signs of degeneration, and the like.

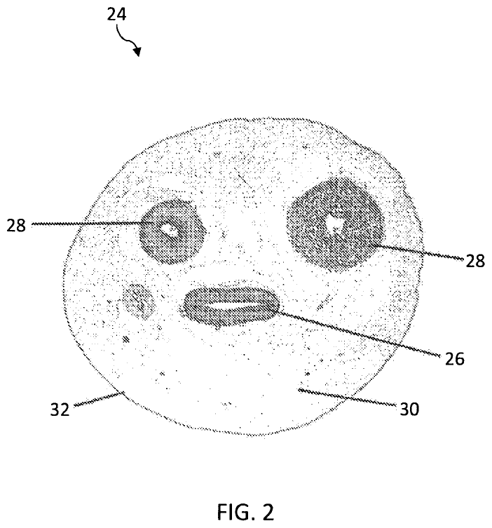

The umbilical cord is a conduit between the developing embryo or fetus and the placenta. As shown in FIG. 2, a cross-section of human umbilical cord 24 is shown. The umbilical cord 24 contains one vein 26, which carries oxygenated, nutrient-rich blood to the fetus, and two arteries 28 that carry deoxygenated, nutrient-depleted blood away. The umbilical cord 24 contains Wharton's jelly 30, a gelatinous substance made largely from mucopolysaccharides located between the amniotic epithelium and the umbilical vessels, which protects the blood vessels inside. The umbilical cord 24 is surrounded by cord lining or umbilical cord lining membrane 32, which is the outermost layer of the umbilical cord 24. The umbilical cord lining membrane 32 comprises two layers: the inner sub-amniotic or mesenchymal layer and the outer amniotic or epithelial layer.

In one embodiment, Wharton's jelly 30 from the umbilical cord is used as the source of mesenchymal stem cells (hUC-MSCs). Wharton's jelly 30 is a gelatinous connective tissue including extracellular matrix abundant in glycosaminoglycans (hyaluronic acid), collagen fibers and myofibroblasts, and sometimes mast cells. Mesenchymal stem cells may be isolated from the Wharton's jelly portion of the umbilical cord. The Wharton's jelly 30 may include variations between the perivascular region, which surrounds the umbilical vein 26 and umbilical arteries 28, the intervascular Wharton's jelly, and the sub-amniotic Wharton's jelly, near the umbilical epithelium 32. The mesenchymal stem cells are multipotent stem cells with a self-renewing capacity and can differentiate into multiple mesenchymal lineages, such as osteocytes, adipocytes, chondrocytes, fibroblasts, etc. Although mesenchymal stem cells derived from Wharton's jelly of a human umbilical cord are exemplified herein, it will be appreciated that other suitable stem cells may be derived from fetal/perinatal tissues, adult tissues, or other sources.

In step 14 of process 10, the mesenchymal stem cells obtained from the umbilical cord (hUC-MSCs) are isolated and/or expanded under hypoxic conditions. To precondition the hUC-MSCs to degenerative host conditions, the stem cells are isolated and expanded in vitro under a hypoxic microenvironment. The hypoxic conditions or microenvironment involves culturing cells under a low oxygen environment with a low oxygen concentration. Hypoxic conditions may include an oxygen concentration (160 mmHg) of 10% or less, 9% or less, 8% or less, 7% or less, 6% or less, 5% less, 4% or less, 3% or less, 2% or less. The oxygen concentration may range from about 1-10% oxygen, about 1-8% oxygen, about 2-8% oxygen, about 1-6%, about 1-5% oxygen, about 2-5% oxygen, or about 1-3% oxygen. The carbon dioxide concentration may be less than 10%, about 1-10% carbon dioxide, about 2-8% carbon dioxide, about 3-7% carbon dioxide, about 4-6% carbon dioxide, or about 5% carbon dioxide. The hypoxic conditions may be similar to or approximate the conditions encountered in degenerative human tissues, for example, typical in degenerative intervertebral discs.

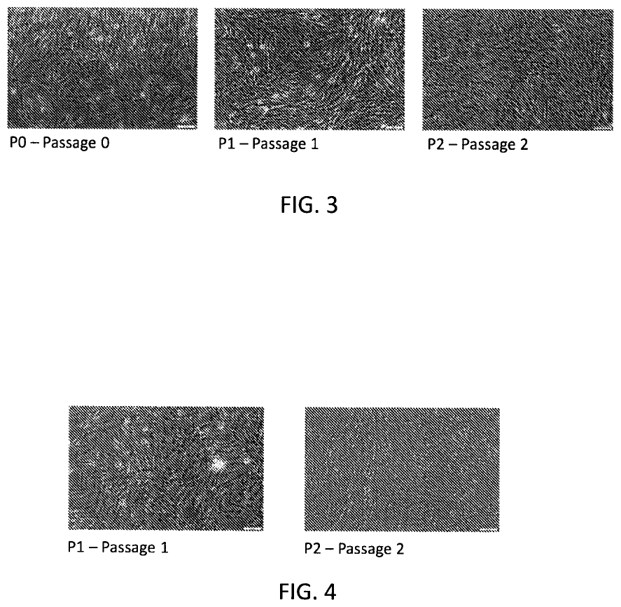

Isolation and expansion of stem cells under hypoxic conditions may occur through one or more cell cultures, splits, or passages. FIG. 3 shows the expansion of stem cells under hypoxic conditions at passage 0 (PO), passage 1 (P1), and passage 2 (P2), respectively. The passage number of the cell culture is a record of the number of times the culture has been subcultured (e.g., harvested and re-seeded), which may increase the population number of available cells. Culture duration may proceed for up to 4 weeks before the stem cells become ready to use as a therapeutic product. Culture duration under hypoxic conditions may range from about 1-10 days, about 4-10 days, about 1 day to 4 weeks, about 1 week to 4 weeks, about 2 weeks to 4 weeks, about 3 weeks to 4 weeks, or another suitable duration. The pre-conditioned cells may be harvested at a passage level up to P5. For example, the pre-conditioned cells may be harvested at a passage level ranging from P1 to P5, P2 to P5, or P3 to P5, or another suitable passage count. It will be appreciated that an appropriate culture duration, including number of passages, seeding densities, and other parameters may be selected to optimize the resulting cell line.

Optionally, the cells obtained from the umbilical cord may also be isolated and expanded under normoxic condition as well. Normoxic conditions include normal oxygen levels in tissue culture flasks, for example, about 20-21% oxygen (160 mmHg). FIG. 4 shows the expansion of stem cells under normoxic conditions for passage 1 (P1) and passage 2 (P2), respectively. If normoxic conditions are used, these cells may be combined with cells preconditioned under hypoxic conditions as described herein.

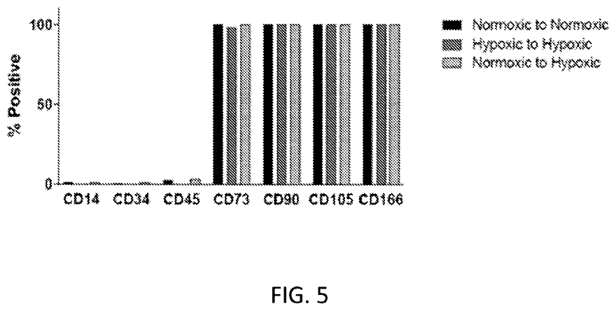

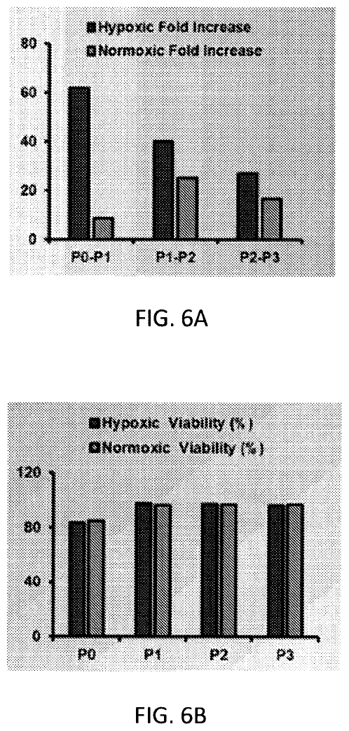

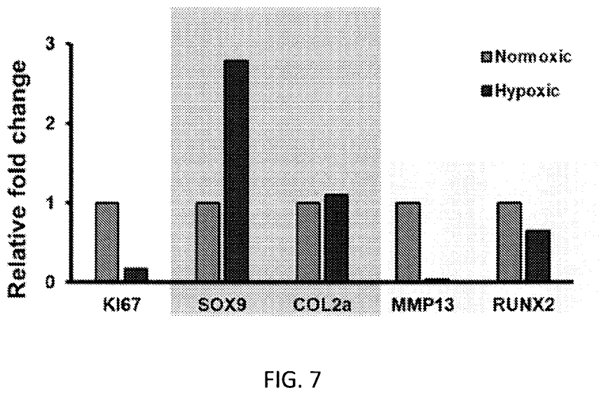

FIGS. 5-7 characterize the pre-conditioned stem cells isolated and/or expanded under hypoxic conditions relative to normoxic conditions. FIG. 5 shows a graph of the flow cytometry characterization for the surface markers (CD14, CD34, CD45, CD73, CD90, CD105, CD166) of the stem cells isolated and expanded in normoxic condition (normoxic to normoxic), hypoxic condition (hypoxic to hypoxic), and isolated in normoxic and expanded in hypoxic (normoxic to hypoxic) condition. The results show that isolation and/or expansion under hypoxic conditions does not affect the phenotype of the stem cells. FIGS. 6A-6B provide a comparative analysis of proliferation of stem cells under hypoxic and normoxic condition at different passage levels (P0, P1. P2, P3). As shown in FIG. 6A, the proliferation of stem cells under hypoxic condition are higher than that under normoxic condition. FIG. 6B shows the viability of stem cells at each passage level was similar among hypoxic and normoxic condition. FIG. 7 provides a real time polymerase chain reaction (PCR) study demonstrating the upregulation of nucleus specific markers (e.g., SOX9 and COL2a) after hypoxic preconditioning compared to normoxic cultured stem cells.

The pre-conditioned cells may be harvested into a single cell suspension, may be processed into micro-spheroids, and/or may include a combination of both. The stem cells may be cultured in an adherent cell culture or a suspension cell culture. In one embodiment, the final cell form may include a single cell suspension. The pre-conditioned cells may be protected in a suspension medium to enhance cell viability and functionality. The suspension medium may include a stabilizer or cryopreservation medium (e.g., freeze media sold under CryoStor® CS10 containing 10% dimethyl sulfoxide (DMSO) or CryoStor® CS5 containing 5% DMSO by Biolife Solutions Inc. of Bothell, WA), human serum albumin, 1% trehalose solution, other suitable suspension medium, or a combination thereof.

In step 18 of process 10, the pre-conditioned cells may be processed into spheroids, and in particular, micro-spheroids. The pre-conditioned mesenchymal stem cells may be aggregated into multi-cellular spheroids. The spheroid production may also occur under hypoxic conditions. The multi-cellular spheroids may include three-dimensional cell cultures arranged during proliferation into sphere-like formations or spherical cellular aggregates. The three-dimensional structure of the micro-spheroids may mimic the natural structure of the implantation site, e.g., the natural phenotype of the nucleus pulposus cells. The structure of the micro-spheroids may also help to minimize potential cell leakage after injection. The spheroids may have aggregates of about 10 to 300 cells, about 20 to 250 cells, about 100 to 250 cells, or about 50 to 200 cells. The micro-spheroids may have sizes ranging from about 10 to 500 μm, about 20 to 400 μm, about 30 to 300 μm, or about 50 to 200 μm. In one embodiment, the cells are available as a micro-spheroid suspension. The suspension medium may be the same as the single cell suspension or may have a different formulation.

In step 20, one or more single cell cultures, micro-spheroids, or both are mixed with one or more carriers. The resultant therapeutic product 22 includes a mixture of pre-conditioned stem cells and one or more carriers. The carrier may affect the overall handling of the material and may influence the efficacy and functionality of the material. In one embodiment, the carrier material is an inactive excipient that acts as an injection medium for the pre-conditioned stem cells. The pre-conditioned cells may be loaded to an anti-or non-angiogenic carrier for injection. The carrier may include a viscous gel or a thermo-responsive gel that can effectively localize the therapeutics at the treatment site. In an exemplary embodiment, the carrier material remains as a viscous liquid during injection to facilitate easy injection and minimal shear stress on the cells during injection. The injectable may be stored, transported, and/or injected at cold temperatures to maintain stability of hUC-MSCs cell viability. Cold temperature may be about 8° C. or less, about 6° C. or less, or may range from about 2-8° C., about 2-6° C., about 3-5° C., or about 4° C. The resulting product may be configured to be injected through 22-27 G needles, for example. In one embodiment, the carrier gel is configured to transition to a thixotropic physical gel at physiological temperature of about 37° C.

Suitable carriers may include, but are not limited to, poloxamers, hydrogels, phospholipids, carboxylmethylcellulose (CMC), glycerin, glycerol, polyethylene glycol (PEG), polylactic acid (PLA), polylactic-co-glycolic acid (PLGA), other copolymers of the same family, and combinations thereof. By way of example, the carrier may include a hydrogel, including a reverse phase hydrogel or temperature sensitive hydrogel, such as hyaluronic acid. Other suitable materials may include gellan gum (GG), methyl cellulose (MC), poloxamer sodium alginate, saline or bone marrow aspirate, for instance.

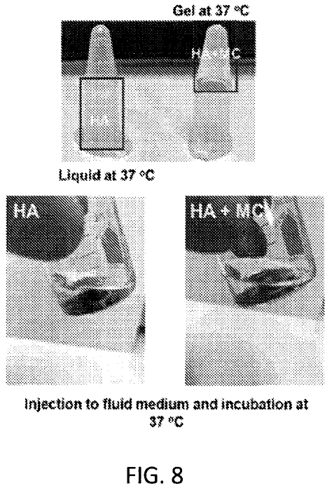

In an exemplary embodiment, the carrier includes a combination of polymeric materials, such as hyaluronic acid (HA), gellan gum (GG), and/or methyl cellulose (MC). The carrier may include hyaluronic acid including high molecular weight hyaluronic acid. The hyaluronic acid may have a molecular weight in the range of 0.9-3 MDa, or 0.94-1.8 MDa. The concentration may range from 0.2% to 2% hyaluronic acid (w/v), 0.5% to 1.5% hyaluronic acid (w/v), 0.8% to 1.2% hyaluronic acid (w/v), or about 1% hyaluronic acid (w/v). As shown in FIG. 8, hyaluronic acid alone may behave as a liquid at physiological temperature (e.g., about 37° C.).

The carrier may include one or more cryoprotectants, such as dimethyl sulfoxide (DMSO), trehalose, propylene glycol, ethylene glycol, or glycerol. The cells and/or the resultant product 22 may be cryopreserved and/or held at cold temperature for storage and transport. The cryoprotectant may be added before freezing or cooling to help protect the cells from damage. DMSO prevents intracellular and extracellular crystals from forming in cells during the freezing process. Trehalose, a sugar molecule, may also enrich the degenerated site with sugar molecules as a supplemented energy source of the resident cells. A suitable cryoprotective agent or method of cryopreservation may be used to preserve the cells and/or resultant product 22 prior to use.

The carrier may include a thermo-gelation agent, such as gellan gum or methyl cellulose. Thermo-gelation agents include carriers that remain liquid at lower temperature (e.g., about 4° C.) and then undergo sol-gel transition at physiological temperature (e.g., about 37° C.). Gellan gum has a thermo-gelation property that may help to localize the cells (and optional hyaluronic acid) to the injection site. Gellan gum may also possess anti-angiogenic properties. If present, the concentration of gellan gum may range from 0.5 to 5% (w/v). A gelation accelerator including a salt such as calcium chloride, sodium chloride, or magnesium chloride may be added to the gellan gum carrier to accelerate the gelation time. For example, in one embodiment, a gelation accelerator such as calcium chloride may be added in the range of 0.01 to 0.5% (w/v).

In one embodiment, the carrier includes methyl cellulose. Methyl cellulose also has a thermo-gelation property. The concentration of methyl cellulose may range from 1 to 12% methyl cellulose (w/v), 2 to 10% methyl cellulose (w/v), 5 to 10% methyl cellulose (w/v), 8 to 10% methyl cellulose (w/v), or about 10% methyl cellulose (w/v). The methyl cellulose viscosity may range from about 15 cP to 400 cP, or about 25cP to 400 cP. In one embodiment, methyl cellulose of about 400 cP (2% solution in water) is used for complete gelation at physiological temperature.

The respective carriers may be combined and concentrations optimized to preserve cell viability, injectability as liquid or viscous liquid, and thermo-gelation at physiological temperature. The carrier components may include a 1:1 mixing of prepared solution or a co-dissolution of the polymers. As shown in FIG. 8, in one embodiment, the carrier may include a combination of hyaluronic acid and methyl cellulose. In an exemplary embodiment, the carrier may include about 1% hyaluronic acid (w/v) and about 5 to 10% methyl cellulose (w/v). Example 1 demonstrates carrier development and optimization with hyaluronic acid and methyl cellulose at varying concentrations. The resulting biomaterial may be injectable as a fluid medium at cold temperature between 2-8° C. (e.g., about 4° C.), which transitions to a gel at physiological temperature (e.g., about 37° C.). This thermoresponsive system effectively administers the treatment effects to the diseased site and prevents leakage and diffusion of stem cells from the injection site.

If desired, other biological agents may be added to the biomaterial. These biological agents may comprise other stem cells, bone morphogenic protein (BMP), peptides, bone growth factors such as platelet derived growth factor (PDGF), vascular endothelial growth factor (VEGF), insulin derived growth factor (IDGF), a keratinocyte derived growth factor (KDGF), or a fibroblast derived growth factor (FDGF), musculoskeletal tissue marrow, and platelet rich plasma (PRP), to name a few. If desired, one or more active pharmaceutical ingredients or medicaments may be incorporated into or added to the biomaterial as well. Biological agents may be added in any suitable pharmaceutically acceptable and effective amounts known in the art.

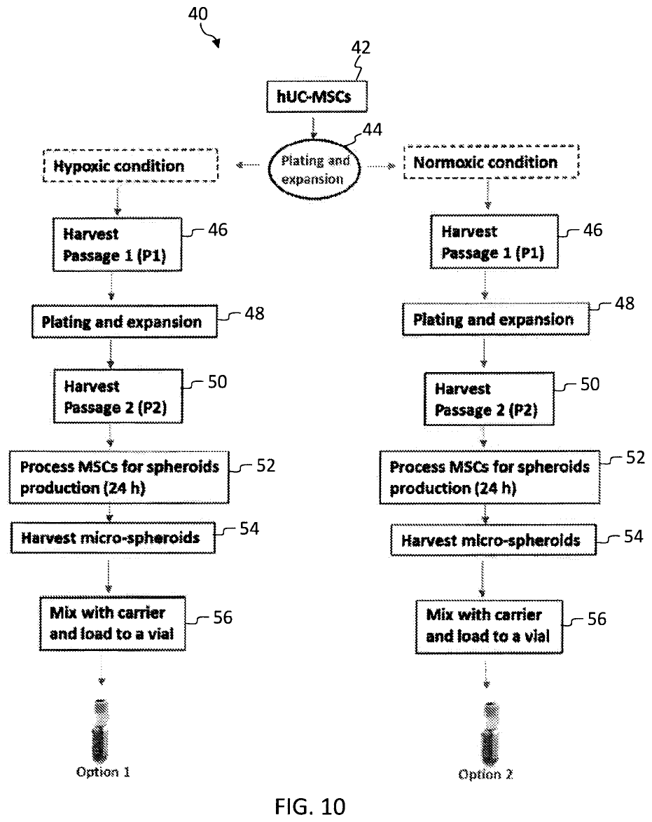

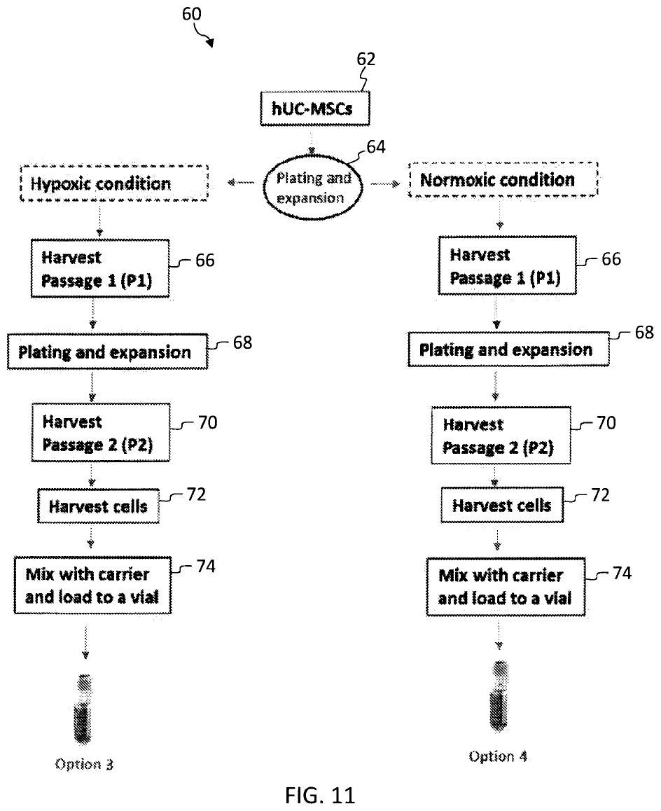

Turning now to FIGS. 10 and 11, flowcharts of processes 40 and 60 for preparing therapeutic products containing viable pre-conditioned stem cells are shown according to further embodiments. In the embodiments shown in FIG. 10, the process 40 may include in vitro conditioning of the stem cells and processing into micro-spheroids. The cells may be developed into micro-spheroids to help minimize potential cell leakage after injection. The three-dimensional structure of the micro-spheroids may also mimic the natural structure of the implantation site, e.g., the natural phenotype of the cells. The process flowchart 40 may include one or more of the following steps: (1) obtaining mesenchymal stem cells 42 from human umbilical cord (hUC-MSCs); (2) plating and expanding the mesenchymal stem cells 44 under hypoxic and/or normoxic conditions; (3) harvesting stem cells 46 at passage 1 (P1); (4) plating and expanding the harvested stem cells 48 from passage 1 (P1); (5) harvesting stem cells 50 at passage 2 (P2); (6) processing the harvested stem cells 52 from passage 2 (P2) into spheroids; (7) harvesting the micro-spheroids 54; and (8) mixing the micro-spheroids 54 with a carrier and loading into a vial 56 to obtain a therapeutic product. The left-hand side of the flowchart 40 shows the steps performed under hypoxic conditions and the right-hand side of the flowchart 40 shows the steps performed under normoxic conditions. It will be appreciated that the spheroids from the hypoxic and normoxic processing may be combined together into the final therapeutic product.

In the embodiments shown in FIG. 11, the process 60 may include in vitro conditioning of the stem cells. The harvested cells may include single cells or a suspension of single cells. The process flowchart 60 may include one or more of the following steps: (1) obtaining mesenchymal stem cells 62 from human umbilical cord (hUC-MSCs); (2) plating and expanding the mesenchymal stem cells 64 under hypoxic and/or normoxic conditions; (3) harvesting stem cells 66 at passage 1 (P1); (4) plating and expanding the harvested stem cells 68 from passage 1 (P1); (5) harvesting stem cells 70 at passage 2 (P2); (6) harvesting stem cells 72; and (7) mixing the harvested cells with a carrier and loading into a vial 74 to obtain a therapeutic product. The left-hand side of the flowchart 60 shows the steps performed under hypoxic conditions and the right-hand side of the flowchart 60 shows the steps performed under normoxic conditions. It will be appreciated that the cells from the hypoxic and normoxic processing may be combined together into the final therapeutic product.

The exogenous stem cells may be expanded in vitro under culture conditions configured to pre-condition the cells. In an exemplary embodiment, the stem cells are isolated and expanded under hypoxic conditions similar to the hypoxic microenvironment encountered in degenerative tissues. The hypoxic pre-conditioning enables the cells to adapt and maintain their functions within the host injection site. The resultant product may remain as a viscous liquid during injection to facilitate easy injection with minimal shear stress on the cells during the injection. Depending on the carrier, the therapeutic product may transition to a gel at physiological conditions to effectively localize the therapeutics to the treatment site and minimize cell leakage.

Although injectable compositions are exemplified herein, it will be appreciated that the biomaterial containing pre-conditioned stem cells may also be formulated into another suitable form. For example, the biomaterial may be in the form of a putty, strip, boat, ring, cylinder, plug, etc. or may be formed into a specific size and shape for a desired application. For example, the biomaterial may be suitable for cervical, thoracic, or lumbar applications and may be suitable for an anterior, posterior, lateral, oblique, anterolateral, transforaminal, or other suitable approach. The biomaterial may be used alone or in combination with a cage, frame, allograft, autograft, graft material, or other biomaterials.

The biomaterials described herein are intended to be applied at a musculoskeletal tissue repair site, e.g., one resulting from degeneration. The biomaterial composition may be injected directly to the treatment site. In an exemplary embodiment, the injectable is formulated for direct injection into the intervertebral disc, facet, or sacroiliac joint. In another exemplary embodiment, the injectable is suitable for treating joint osteoarthritis of the knee, hip, shoulder, rotator cuff, etc. It will be appreciated that the biomaterial may be utilized in a wide variety of orthopedic, periodontal, neurosurgical, oral and maxillofacial surgical procedures. Possible clinical applications may include e.g., the treatment of spinal disc degeneration or disease, traumatic, pathologic, treatment of osteoarthritis of the knee, hip, shoulder, etc. or other defects in any soft tissue, musculoskeletal tissues of the body.

The term “treating” and the phrases “treatment of a disease” and “treatment of a condition” refer to executing a protocol that may include the use of the compositions and methods herein and/or administering one or more biomaterials to a patient (human, normal or otherwise, or other mammal), in an effort to alleviate signs or symptoms of the disease or condition. Alleviation can occur prior to signs or symptoms of the disease or condition appearing, as well as after their appearance. Thus, “treating” or “treatment” includes “preventing” or “prevention” of disease or undesirable condition. In addition, “treating” or “treatment” does not require complete alleviation of signs or symptoms and does not require a cure to the ailment.

Further example embodiments are directed to kits that include components for making and storing the present biomaterials including, for example, prepared stem cells, carriers, and the like, and bottles or vials for storage. The kit may include components for administering the biomaterial to the patient including needles, syringes, or other dispensers. It is further contemplated that the kit may include other components, such as scaffolds, cages, spacers, bone graft materials, or the like. Additional components, instructions and/or apparatus may also be included.

The following examples are provided to further illustrate various non-limiting embodiments and techniques. It should be understood, however, that these examples are meant to be illustrative and do not limit the scope of the claims. As would be apparent to skilled artisans, many variations and modifications are intended to be encompassed within the spirit and scope of the invention.

### EXAMPLES

### Example 1

This example demonstrates the carrier development and optimization with hyaluronic acid and methyl cellulose. As shown in the table below, solutions were prepared with hyaluronic acid and methyl cellulose at different concentrations. The methyl cellulose was also provided at two different viscosities. In the 1:1 mixing of the solutions, liquid to highly viscous liquids were observed at preservation and injection temperature (4° C.). However, no gelation was observed at physiological temperature (37° C.). With the co-dissolution of the polymers, viscous liquids were observed at preservation and injection temperature (4° C.) and gelation was observed at physiological temperature (37°° C.) for the higher concentrations of methyl cellulose.

1:1 Mixing of Prepared Solution

| | Methyl | | |
|---|---|---|---|
| Hyaluronic | cellulose | Observation at | Observation |
| acid | (15 cP) | 4° C. | at 37° C. |
| 1% | 1% | Liquid | No gelation |
| 1% | 2% | Viscous liquid | No gelation |
| 1% | 5% | Viscous liquid | No gelation |
| 1% | 10% | Highly viscous | No gelation |
| | | liquid | |
| 1% | 10% in | Highly viscous | No gelation |
| | 0.3% CaCl₂ | liquid | |

| | Methyl | | |
|---|---|---|---|
| Hyaluronic | cellulose | Observation at | |
| acid | (400 cP) | 4° C. | Observation at 37° C. |
| 1% | 2% | Viscous liquid | No gelation |
| 1% | 5% | Viscous liquid | No gelation |

Co-Dissolution of Polymers

| | | | |
|---|---|---|---|
| 1% | 2% | Viscous liquid | Incomplete gelation |
| 1% | 5% | Viscous liquid | Complete gelation |
| | | | but longer |
| | | | incubation needed |
| 1% | 10% | Viscous liquid | Complete gelation |

### Example 2

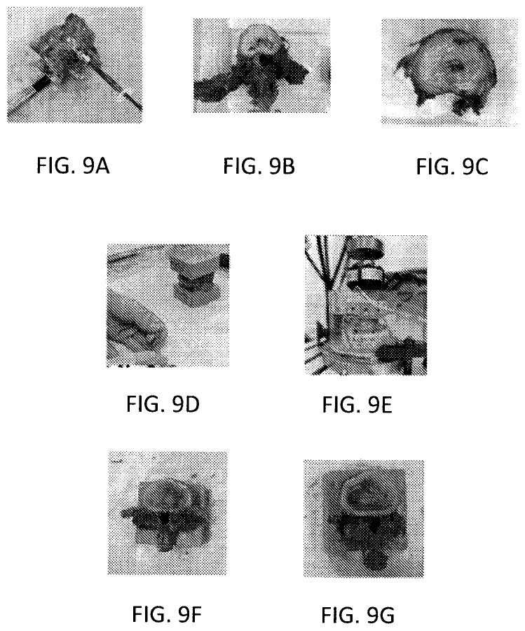

This example demonstrates injection of carrier material to an ex vivo spine under static and mechanical loading. The results showed localized dispersion of gel material within the spine with minimal dispersion under static condition. FIGS. 9A-9G show injection to the ex vivo spine and subsequent evaluation. FIG. 9A shows injection of the carrier material into the ex vivo spine segment. The biomaterial was directly injected into the intervertebral disc between adjacent vertebrae with a syringe. FIGS. 9B-9C show axial views of the intervertebral disc including the carrier material injected therein. The gelled carrier material remained localized in the treatment site. The spine segment was then subjected to static and mechanical loading. FIGS. 9D-9E show the instrumentation for analyzing the mechanical forces. The analyzer applied one or more mechanical forces to the spine segment, such as torsional and/or axial forces, to determine the stress and strain on the spine segment. The resulting intervertebral discs are shown in FIGS. 9F-9G. The results showed localized dispersion of gel material within the spine with minimal dispersion under static conditions.

It will be further understood that various changes in the details, materials, and arrangements of the parts which have been described and illustrated in order to explain the nature of this invention may be made by those skilled in the art without departing from the scope of the invention as expressed in the claims. One skilled in the art will appreciate that the embodiments discussed above are non-limiting. It will also be appreciated that one or more features of one embodiment may be partially or fully incorporated into one or more other embodiments described herein.

What is claimed is:

1. A biomaterial comprising:
human umbilical cord mesenchymal stem cells preconditioned under a hypoxic environment; and
a thermo-responsive carrier being a viscous liquid at cold temperature between 2-8° C. and a gel at physiological temperature of 37°° C.

2. The biomaterial of claim 1, wherein the thermo-responsive carrier includes hyaluronic acid, gellan gum, methyl cellulose, or a combination thereof.

3. The biomaterial of claim 1, wherein the thermo-responsive carrier includes a co-dissolution of hyaluronic acid and methyl cellulose.

4. The biomaterial of claim 1, wherein the thermo-responsive carrier includes 0.2% to 2% (w/v) hyaluronic acid and 2-10% (w/v) methyl cellulose.

5. The biomaterial of claim 1, wherein the human umbilical cord mesenchymal stem cells include a single cell suspension.

6. The biomaterial of claim 1, wherein the human umbilical cord mesenchymal stem cells include a suspension of micro-spheroids having 50-200 cells with sizes ranging from 50 to 200 μm.

7. The biomaterial of claim 1, wherein the hypoxic conditions include an oxygen concentration of 10% or less.

8. The biomaterial of claim 1, wherein the hypoxic conditions include an oxygen concentration ranging from 1-6%.

9. The biomaterial of claim 1, wherein the human umbilical cord mesenchymal stem cells are obtained from Wharton's jelly of a human umbilical cord.

10. A method for manufacturing a biomaterial for aiding musculoskeletal tissue regeneration, the method comprising:
obtaining human umbilical cord;
isolating viable stem cells from the umbilical cord;
expanding the viable stem cells under hypoxic conditions; and
combining the expanded viable stem cells with an inert carrier to form a therapeutic product.

11. The method of claim 10, wherein the hypoxic conditions include an oxygen concentration of 10% or less.

12. The method of claim 10, wherein the hypoxic conditions include an oxygen concentration ranging from 1-6%.

13. The method of claim 10, wherein the viable stem cells are mesenchymal stem cells.

14. The method of claim 10, wherein the expanded viable stem cells include a single cell suspension, suspension of micro-spheroids, or a combination of both.

15. The method of claim 10, wherein the viable stem cells are obtained from Wharton's jelly of a human umbilical cord.

16. The method of claim 10, wherein the inert carrier includes a thermo-gelation agent.

17. The method of claim 10, wherein the inert carrier includes hyaluronic acid, gellan gum, methyl cellulose, or a combination thereof.

18. A method of promoting musculoskeletal tissue and/or soft tissue healing in a mammal, the method comprising:
providing a biomaterial including human umbilical cord mesenchymal stem cells preconditioned under a hypoxic environment; and
administering the biomaterial into a target repair site to facilitate repair or regeneration of musculoskeletal tissue and/or soft tissue at the target repair site.

19. The method of claim 18, wherein the biomaterial is injected into the target repair site as a liquid and transitions to a gel at physiological temperature.

20. The method of claim 18, wherein the target repair site is a defect or injury in the spine.
